(19) United States
(12) Patent Application Publication
(10) Pub. No.: US 2024/0149042 A1
(43) Pub. Date: May 9, 2024

(54) MEDICINAL PRODUCT, FUNCTIONAL PART FOR A MEDICINAL PRODUCT, AND METHOD FOR STERILIZING AND/OR FOR PRODUCING STERILIZATION STABILITY IN A MEDICINAL PRODUCT OR FUNCTIONAL PART

(71) Applicant: B. Braun Melsungen AG, Melsungen (DE)

(72) Inventors: Liesa Glaess, Schuepfheim (CH); Benjamin Widmer, Langnau (CH); Manuel Arnaldi, Ruederswil (CH)

(21) Appl. No.: 18/280,017

(22) PCT Filed: Feb. 25, 2022
(86) PCT No.: PCT/EP2022/054802

§ 371 Date: Sep. 1, 2023

(30) Foreign Application Priority Data
Mar. 3, 2021 (DE) 10 2021 202 061.5

(51) Int. Cl. A61M 39/24

(52) U.S. Cl. CPC A61M 39/24; A61M 2039/2426; A61M 2205/0216; A61M 2207/10

(57) ABSTRACT

A medical device, an elastically deformable functional part for a medical device, and a method for sterilizing and/or for establishing sterilization resistance of a medical device or an elastically deformable functional part. The elastically deformable functional part has an openable slit arrangement. The slit arrangement widens or opens upon elastic deformation of the elastically deformable functional part and recloses upon cessation of the elastic deformation. The slit arrangement has slit wall faces with a separating agent. The separating agent can be a solid.

### CROSS-REFERENCE TO RELATED APPLICATIONS

This application is the United States national stage entry of International Application No. PCT/EP2022/054802, filed on Feb. 25, 2022, and claims priority to German Application No. 10 2021 202 061.5, filed on Mar. 3, 2021. The contents of International Application No. PCT/EP2022/054802 and German Application No. 10 2021 202 061.5 are incorporated by reference herein in their entireties.

### FIELD

The invention relates to a medical device comprising an elastically deformable functional part having an openable slit arrangement, to a corresponding functional part for a medical device and to a method for sterilizing and/or for establishing sterilization resistance of such a medical device or functional part.

### BACKGROUND

Gamma irradiation is a common sterilization method for medical devices. If the medical devices comprise a radiation-crosslinkable material, such as silicone elastomers, these materials will react with crosslinking reactions when exposed to high-energy radiation. In the case of slit valves, this can lead to an undesirable closing up of the slits, which is also referred to as healing.

In order to prevent such healing of slits, additives are usually added. One disadvantage is that such additivation generally leads to opaque materials. However, for many applications, for example connection devices in the field of medical infusion therapy, the maintenance of transparency of individual functional parts, such as valve bodies, is absolutely necessary for the intended use. Another problem is that the addition of solid additives can cause fundamental changes in material properties.

In addition to solid additives, liquid additives are also used. However, they have to have sufficient wettability and, in particular, steadfastness in order to be able to perform their function. Radiation sterilization, ageing processes and transport processes generally involve elevated temperatures, as a result of which the separation effect of liquid separating agents over time is frequently found to be too low. In addition, some liquid separating agents, for example silicone oils, also react to radiation sterilization, as a result of which the separating action may be additionally diminished.

### SUMMARY

It is therefore an object of the present invention to provide a medical device and a functional part for a medical device that partially or completely prevents disadvantages known in connection with medical devices or functional parts of the type in question, in particular closing up of slits and/or loss of transparency. It is a further object of the invention to provide a method for sterilizing and/or for establishing sterilization resistance of a medical device or functional part for a medical device that partially or completely circumvents the disadvantages mentioned at the start.

According to a first aspect, the invention relates to a medical device.

The medical device comprises an elastically deformable functional part having an openable slit arrangement, wherein the slit arrangement widens, opens or opens out upon elastic deformation of the elastically deformable functional part and recloses upon cessation of the elastic deformation. Preferably, the manner in which the slit arrangement widens upon the elastic deformation of the elastically deformable functional part or the slit arrangement opens upon the elastic deformation of the elastically deformable functional part is such that said slit arrangement allows a flow of a fluid, in particular a liquid, preferably medical solution, or a gas, through the elastically deformable functional part. Elastic deformation of the elastically deformable functional part can occur, for example, when connecting the medical device to an infusion syringe, a flexible tube or flexible tube system or the like, especially of a medical infusion system.

The medical device is particularly distinguished by the fact that slit wall faces of the slit arrangement comprise a separating agent or are provided with a separating agent comprising a solid, preferably biocompatible solid, or consisting of a solid, preferably biocompatible solid. Preferably, the slit wall faces, in particular only the slit wall faces, of the slit arrangement are coated with the separating agent at least partially, in particular only to partially or completely, i.e. across the entire surface or continuously.

In a closed position of the slit arrangement, the slit wall faces are preferably arranged or positioned next to one another at least sectionally, in particular only sectionally or, this being preferred, continuously, in particular along a fluid passage axis, in particular central longitudinal axis, of the elastically deformable molding. In an open position of the slit arrangement, the slit wall faces or slit walls are preferably pushed apart, for example by an infusion syringe, a flexible tube or a flexible tube system or the like, especially of a medical infusion system.

In the context of the present invention, the expression “functional part” is to be understood to mean a component or a component part or a construction element of the medical device.

In the context of the present invention, the expression “slit wall faces” is to be understood to mean faces or surfaces of walls that form or define a slit or are involved in the formation of a slit.

In the context of the present invention, the expression “separating agent” is to be understood to mean a substance capable of reducing or (completely) avoiding closing up of slits, in particular that induced by radiation.

In the context of the present invention, the expression “biocompatible solid” is to be understood to mean a solid which can be used in the field of medicine, in particular in medical fields with direct or indirect patient contact, without health concerns or risks.

The invention is based on the surprising finding that solids, preferably biocompatible solids, are suitable as separating agents for preventing healing phenomena, i.e. closing up of slits, in elastically deformable functional parts and medical devices comprising such functional parts. Upon exposure to radiation, in particular gamma radiation, beta radiation, X-radiation or electron beam radiation (E-beam radiation), better protection against radiation-induced crosslinking phenomena is thus achievable. It is especially advantageous that the invention makes it possible to carry out radiation sterilization, in particular gamma radiation sterilization, with a radiation dose of at least 32 kGy, without (appreciable) closing up of slits. Furthermore, it is advantageous that an increase in ageing resistance is achievable by the invention.

The separating agent is preferably liquid-free, i.e. free of liquids. As a result, the disadvantages described at the start in connection with liquid additives can be avoided.

In an embodiment of the invention, the functional part is at least sectionally transparent, i.e. light-transmissive, in particular only sectionally or completely transparent. In the context of the present invention, the expression “transparent” or “light-transmissive” is to be understood to mean the ability of matter to allow electromagnetic waves having a wavelength of 380 nm to 780 nm to pass through (transmission). In this respect, the invention is based on the further surprising finding that, in the case of a transparent functional part, the separating agent additivation envisaged by the invention does not lead to an opaque change in the functional part. Instead, the transparency of the elastically deformable functional part is advantageously maintained.

In a further embodiment of the invention, the elastically deformable functional part comprises an elastically deformable material, in particular a transparent, i.e. light-transmissive, elastically deformable material, or the elastically deformable functional part, in particular as one piece, is made of an elastically deformable material, in particular transparent, i.e. light-transmissive, elastically deformable material.

The elastically deformable material is preferably an elastomer and/or thermoplastic elastomer.

The elastomer may in particular be selected from the group consisting of silicone elastomers such as silicone rubber, acrylonitrile-butadiene rubber, hydrogenated acrylonitrile-butadiene rubber and mixtures of at least two of the aforementioned elastomers. Alternatively or in combination, the elastomer may be another polymer which is crosslinking-capable or in which crosslinking reactions predominate.

In a further embodiment of the invention, the slit wall faces, in particular only the slit wall faces, of the slit arrangement are coated, in particular powder-coated or wetted, with the separating agent at least partially, in particular only partially or completely, i.e. continuously or across the entire surface. Preferably, the elastically deformable functional part, apart from the slit wall faces of the slit arrangement, is free of the separating agent. In particular, the separating agent is not present, in particular not dispersed or distributed, within the elastically deformable functional part. Instead, the separating agent is preferably exclusively present on the slit wall faces of the slit arrangement. This can advantageously additionally reduce the risk that material properties, such as in particular transparency and/or mechanical properties, of the functional part change.

In a further embodiment of the invention, the solid is in the form of a powder. As a result, the separating agent can be applied to or incorporated into the slit wall faces or between the slit wall faces of the slit arrangement, for example by means of compressed air, in a complication-free manner. This in turn contributes to an optimal separating action of the separating agent.

In a further embodiment of the invention, the solid, in particular the powder, has a particle size or an average particle diameter of <(in words: smaller than) 400 μm, in particular 1 μm to 50 μm, preferably 0.1 μm to 10 μm. As a result, application can be additionally optimized.

In a further embodiment of the invention, the solid is a salt. In particular, the solid, in particular salt, may be an alkali metal salt, in particular selected from the group consisting of alkali metal chloride, alkali metal bromide, alkali metal iodide and mixtures of at least two of the aforementioned alkali metal salts.

The alkali metal chloride may in particular be selected from the group consisting of lithium chloride, sodium chloride, potassium chloride and mixtures of at least two of the aforementioned alkali metal chlorides.

The alkali metal bromide may in particular be selected from the group consisting of lithium bromide, sodium bromide, potassium bromide and mixtures of at least two of the aforementioned alkali metal bromides.

The alkali metal iodide may in particular be selected from the group consisting of lithium iodide, sodium iodide, potassium iodide and mixtures of at least two of the aforementioned alkali metal iodides.

Particularly preferably, the solid is sodium chloride.

When using aforementioned salts, in particular sodium chloride, the advantages of the invention can be realized particularly well.

In a further embodiment of the invention, the solid is a saccharide. The saccharide may in principle be a monosaccharide, oligosaccharide, such as disaccharide, or polysaccharide, or a mixture of at least two of the aforementioned saccharides.

Preferably, the solid, in particular saccharide, is glucose.

By using a saccharide, in particular glucose, the advantages of the invention are (likewise) highly pronounced.

In a further embodiment of the invention, the solid is a vitamin, in particular ascorbic acid, preferably L-(+)-ascorbic acid, i.e. vitamin C. The advantages of the invention can be (also) realized particularly well when using ascorbic acid, preferably L-(+)-ascorbic acid or vitamin C.

It may further be preferable that the solid is in the form of a mixture. The mixture comprises preferably at least two substance or consists of at least two substances selected from the group consisting of salts, saccharides, vitamins and mixtures of at least two of said substances. Particularly preferably, the mixture may comprise at least two substances or consist of at least two substances selected from the group consisting of sodium chloride, glucose and L-(+)-ascorbic acid (vitamin C). Accordingly, the mixture may comprise for example sodium chloride and glucose or consist of sodium chloride and glucose. Alternatively, the mixture may comprise for example sodium chloride and L-(+)-ascorbic acid or consist of sodium chloride and L-(+)-ascorbic acid. Alternatively, the mixture may comprise for example glucose and L-(+)-ascorbic acid or consist of glucose and L-(+)-ascorbic acid. Alternatively, the mixture may comprise for example sodium chloride, glucose and L-(+)-ascorbic acid or consist of sodium chloride, glucose and L-(+)-ascorbic acid.

In addition, the separating agent may comprise a liquid as well as the solid. This can advantageously improve not only the wettability of the slit wall faces of the slit arrangement, but also the adhesion of the separating agent to the slit wall faces of the slit arrangement. In particular, the solid may for this purpose be wetted with an appropriate liquid. The liquid may for example be an oil or a mixture of oils.

In a further embodiment of the invention, the slit arrangement is in the form of a longitudinal slit arrangement, i.e. linear slit arrangement.

Alternatively, the slit arrangement may deviate from this, for example may be cross-shaped, i.e. may be in the form of a so-called cross-slit arrangement.

In a further embodiment of the invention, the elastically deformable functional part is in the form of a valve body.

In a further embodiment of the invention, the elastically deformable functional part, in particular the valve body, has a head region, in particular a head region in lid form. The slit arrangement is preferably formed along a transverse extent, i.e. transverse to a longitudinal extent, of the head region. Particularly preferably, the slit arrangement extends, in particular centrally, along a longitudinal axis or fluid passage axis, in particular the central longitudinal axis, through the head region. Preferably, a casing of the elastically deformable functional part adjoins the head region, in particular directly. If the elastically deformable functional part is in the form of a valve body, this being preferred according to the invention, a valve casing adjoins the head region, in particular directly. The valve casing preferably has a radial cross-section which preferably has a larger longitudinal extent than transverse extent. The radial cross-section of the valve casing is based on a longitudinal axis or fluid passage axis of the valve body, which preferably corresponds to a central longitudinal axis of the valve body. The head region and/or casing, for example only the head region or only the casing, of the elastically deformable functional part, in particular valve body, may be transparent, i.e. may comprise a transparent, elastically deformable material, preferably an elastomer, in particular silicone elastomer, and/or thermoplastic elastomer, or may be made of such a material. Regarding further suitable materials, reference may be made to the previous description.

Preferably, the elastically deformable functional part is used for a connection device of a medical infusion system. A relevant connection device is known, for example, from EP 3 216 486 A1. The features and advantages described there in relation to the connection device are incorporated in the present description by express reference.

In a further embodiment of the invention, the medical device is a medical device for infusion therapy. Especially preferably, the medical device is a connection device, especially of a medical infusion system. The medical device, in particular the connection device, may be, for example, in the form of a one-way valve, two-way valve, three-way valve or four-way valve or in the form of a valve manifold, i.e. in the form of a unit or system having or composed of one-way valves arranged or connected one after the other, i.e. in series. Preferably, the medical device, in particular the connection device, is in the form of a two-way valve, three-way valve or four-way valve or in the form of a valve manifold.

Preferably, the medical device is in sterilized form, in particular radiation-sterilized form, preferably gamma radiation-sterilized, beta radiation-sterilized, x-radiation-sterilized or electron beam-sterilized form. Preferably, the medical device is sterilized by irradiation, preferably gamma irradiation, beta irradiation, X-ray irradiation or electron beam irradiation (E-beam irradiation), in particular with a radiation dose of at least 32 kGy, in particular 32 kGy to 100 kGy, in particular 32 kGy to 45 kGy.

Alternatively, the medical device may be in non-sterilized form.

According to a second aspect, the invention relates to an elastically deformable functional part for a medical device.

The elastically deformable functional part comprises an openable slit arrangement which widens, opens or opens out upon elastic deformation of the elastically deformable functional part and recloses upon cessation of the elastic deformation. Slit wall faces of the slit arrangement comprise a separating agent that comprises a solid, preferably biocompatible solid, or consists of a solid, preferably biocompatible solid.

Regarding further features and advantages of the elastically deformable functional part, in particular the slit arrangement and the separating agent, and of the medical device, full reference is made to the corresponding statements made under the first aspect of the invention. The features and advantages described there in this respect also apply mutatis mutandis to the elastically deformable functional part according to the second aspect of the invention.

According to a third aspect, the invention relates to a method for sterilizing and/or for establishing sterilization resistance of a medical device according to the first aspect of the invention or of an elastically deformable functional part according to the second aspect of the invention. The method comprises, in chronological order, the following steps:
a) coating or loading, in particular powder-coating or wetting, the slit wall faces of the slit arrangement with the separating agent comprising a solid, preferably biocompatible solid, or consisting of a solid, preferably biocompatible solid, and
b) sterilizing the medical device or elastically deformable functional part, in particular by exposure to radiation, preferably gamma radiation, X-radiation, beta radiation or electron beam radiation (E-beam radiation).

The method according to the third aspect of the invention is thus distinguished by the fact that step b) is carried out after step a) has been carried out.

The slit wall faces of the slit arrangement may be coated or loaded, in particular powder-coated or wetted, with the separating agent only partially or completely, i.e. continuously or across the entire surface.

Furthermore, step a) may be carried out with compressed air, which transports the separating agent to the slit wall faces of the slit arrangement. For this, step a) may be carried out at an air pressure of 0.1 bar to 7 bar, preferably 0.5 bar to 3 bar. Furthermore, step a) may be carried out with an air volume flow time of 1 ms (0.001 s) to 5 s, preferably, 50 ms (0.05 s) to 1 s.

Preference is given to using a salt as the solid, in particular an alkali metal salt, preferably an alkali metal chloride, particularly preferably sodium chloride. With regard to further possible salts, reference is made to the corresponding salts disclosed under the first aspect of the invention. The salts additionally described there may likewise be used in the context of the method according to the invention.

Alternatively, preference is given to using a saccharide as the solid, in particular a monosaccharide, oligosaccharide (such as a disaccharide) or polysaccharide. Particular preference is given to using glucose as a biocompatible solid.

Alternatively, preference is given to using a vitamin as the solid, in particular ascorbic acid, particularly preferably L-(+)-ascorbic acid (vitamin C).

Furthermore, step b) may be carried out with a radiation dose of at least 32 kGy, in particular 32 kGy to 100 kGy, in particular 32 kGy to 45 kGy.

Regarding further features and advantages of the method, full reference is made to the corresponding statements made under the first and second aspects of the invention. The features and advantages described there especially in relation to the elastically deformable functional part and to the medical device also apply mutatis mutandis to the method according to the third aspect of the invention.

Further features and advantages of the invention will become apparent from the following description of preferred embodiments with reference to figures and FIG. descriptions. Here, individual features of the invention can be realized singly or plurally in combination with one another. The preferred embodiments merely serve to elucidate the invention, which is not to be limited thereto in any way.

### BRIEF DESCRIPTION OF THE DRAWINGS

FIG. 1 shows a plan view of one embodiment of a medical device according to the invention in the form of a connection device;

FIG. 2 shows a section through the medical device according to FIG. 1 along the section line II-II in FIG. 1;

FIG. 3 shows a view from below of a functional part of the medical device according to FIG. 2 in the form of a valve body;

FIG. 4 shows a longitudinal section through the valve body according to FIG. 3 along the section line Iv-Iv in FIG. 3;

FIG. 5 shows a further longitudinal section through the valve bodies along the section line V-V in FIG. 3;

FIG. 6 shows a plan view of the valve body according to FIGS. 3 to 5;

FIGS. 7 to 9 show sectional views of different steps during connection of a component to the medical device according to FIG. 2; and

FIGS. 10a and 10b show one embodiment of a method according to the invention.

### DETAILED DESCRIPTION

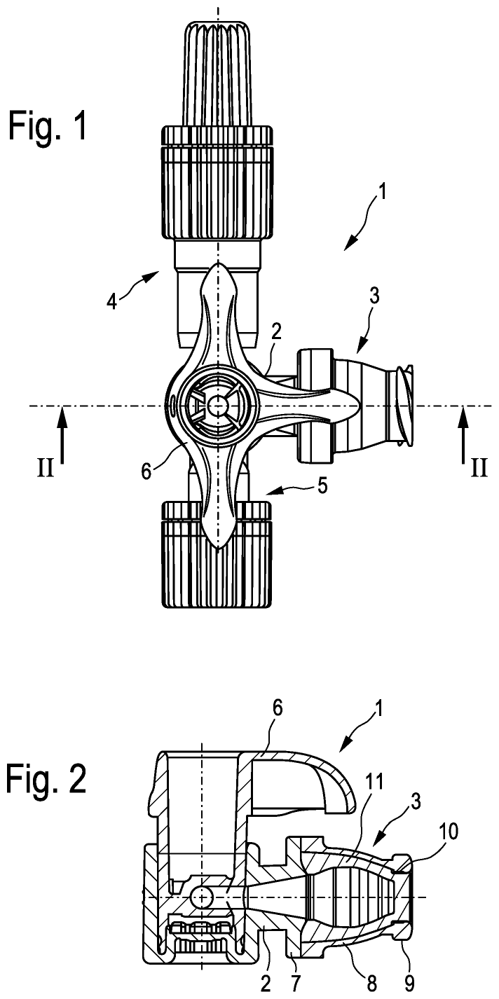

FIG. 1 shows schematically one embodiment of a medical device according to the invention in the form of a connection device 1 of a medical infusion system. The connection device 1 may, for example, be in the form of a three-way valve, as shown in FIG. 1. It will be appreciated, however, that the connection device 1 may deviate from this, in particular may be in the form of a one-way or two-way valve or in the form of a valve manifold, i.e. in the form of a unit or system having or composed of one-way valves connected in series, i.e. one after the other.

The connection device 1 comprises a housing 2, a connection nozzle 3 and two connection regions 4, 5. Rotatably mounted in the housing 2 is an actuator 6. Further provided in the housing 2 are a total of three connection channels which are blocked or connected to each other depending on the position of the actuator 6. One connection channel of the housing 2 leads to the connection nozzle 3. A further connection channel arranged at a right angle leads to the connection region 4 and, opposite to this, a third connection channel leads to the connection region 5. One of the two connection regions 4, 5 is intended for the connection of a patient line. The other connection region 4, 5 is used to connect a connection line to a fluid container.

The connection nozzle 3 is preferably provided for temporary connection of a component of the medical infusion system, such as in particular a syringe, in order to supply the patient line with, for example, (additional) medicaments or the like. Additionally or alternatively, the connection nozzle 3 may be provided for connection, in particular continuous connection (permanent connection), of an additional line.

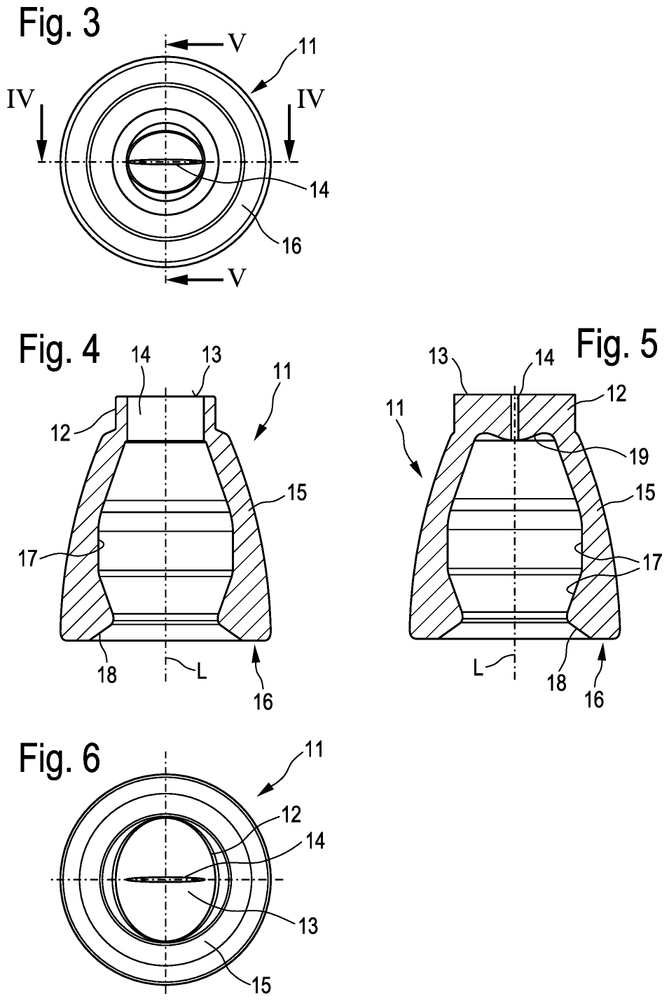

The connection nozzle 3 will be more particularly elucidated with reference to FIGS. 3 to 5 that follow.

The connection nozzle 3 may have a dimensionally stable cap 8 which, at its end face that faces the housing 2, is connected, preferably fixedly connected, to a dimensionally stable base section 7 of the housing 2. The cap 8 is sleeve-shaped and has, on the side facing the base section 7, a thickened edge region which is fixedly connected to the base section 7, for example by welding. The base section 7 is dish-shaped and protrudes radially outwards relative to a central longitudinal axis L of the connection nozzle 3. The base section 7 surrounds a channel section which conically tapers towards an interior of the housing 2.

At its end region that is remote from the base section 7, the cap 8 is provided with a passage 10 which is closable by a valve body 11 of the connection device 1 that will be described in greater detail below. The passage 10 is enclosed by a thickened edge region which is provided with connection profiling 9 in the form of Luer-lock profiling.

The valve body 11 is preferably cup-shaped or bell-shaped and in particular made of an elastically deformable material as a single piece. The elastically deformable material is preferably an elastomer or thermoplastic elastomer. Particularly preferably, the elastically deformable material is a silicone elastomer, such as silicone rubber. Preferably, the elastically deformable material is moreover transparent, i.e. light-transmissive.

The valve body 11 preferably has an outer contour which, in an unloaded, i.e. non-deformed, initial state, is in flush and planar contact with the inner contour of the cap 8 over a total height of the cap 8. The valve body 11 has a head region 12, in particular a head region in lid form. The head region 12 may have a rotationally symmetrical, in particular circular, or, as depicted, a rotationally unsymmetrical, in particular oval, main face (see in particular FIGS. 3 and 6). The valve body 11 further comprises a valve casing 15. The valve casing 15 adjoins the head region 12, preferably directly. Preferably, the valve casing 15 is provided with a base ring 16 at a lower end-face region. The head region 12 is provided with a slit arrangement 14. A surface 13 of the head region 12 is preferably smooth and flat.

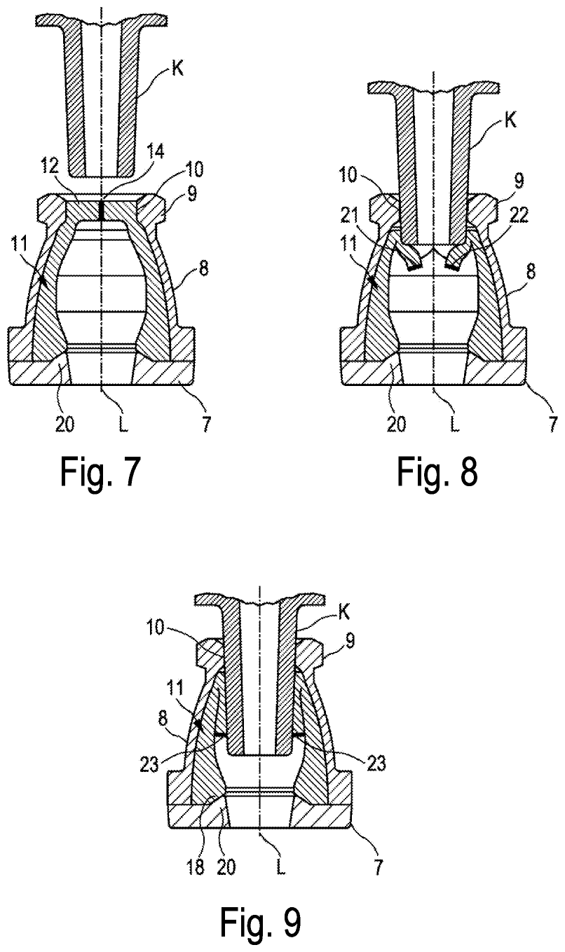

From FIG. 7, it can be seen that, in an unloaded initial state of the valve body 11, the surface 13 of the head region 12 can be flush in the cap 8 with a marginal edge of the passage 10. From said marginal edge of the passage 10, an oblique chamfer extends outwards up to an end face of the edge region of the cap 8 that defines the passage 10. Consequently, in the unloaded initial state of the valve body 11, the end face of the edge region of the cap 8 including the surface 13 of the head region 12 of the valve body 11 can be cleaned and disinfected in a simple manner, for example by means of a disinfectant cloth or the like, by medical personnel.

The valve casing 15 of the valve body 11 is preferably rotationally symmetrical relative to the central longitudinal axis L. Preferably, the valve casing 15 has a wall which thickens starting from the head region 12 up to the base region 16. Said thickening may occur discontinuously and non-linearly, as can be seen from the two visible edges shown. The edges are preferably annularly circumferential. The valve casing 15 may have a first wall section of constant thickness that adjoins the head region 12 and expands like a truncated cone. Adjoining said first wall section, in the direction of the base ring 16, may be a second wall section, the inner wall of which runs cylindrically and coaxially in relation to the central longitudinal axis L and the outer wall of which runs bulgingly further outwards towards the base ring 16. Adjoining said wall section, in particular central wall section, may be a base-side wall section. Preferably, the base-side wall section comprises the base ring 16. Preferably, in this region, the inner wall tapers towards the base section starting from the cylindrical central region, thereby yielding an inner wall section which conically tapers downwards.

The inner wall may conically expand again towards the end face of the base ring 16 to form a contact surface 18. Consequently, an egg-shaped or O-shaped inner contour 17 can result over the height of the valve casing 15 (see FIGS. 4 and 5).

An inner face of the head region 12 pointing into the interior of the valve casing 15 may be a dome-shaped contour 19, as shown in FIG. 5.

The passage 10 of the cap 8 may be rotationally symmetrical or rotationally unsymmetrical. In the case of a rotationally unsymmetrical head region 12, the passage 10 is preferably also rotationally unsymmetrical in a complementary manner.

As can be seen from FIGS. 2 and 7 to 9, the conical contact surface 18 of the base ring 16 of the valve body 11 is assigned a complementarily conical support face 20 in the region of the base section 7, as a result of which the valve body 11 is planarly supported on the base section 7 in the region of the base ring 16 over its entire radial width.

The slit arrangement 14 is oriented transversely to a longitudinal extent of the head region 12, as can be seen from FIGS. 4 to 6. The slit arrangement 14 preferably extends centrally along the central longitudinal axis L through the head region 12. The slit arrangement 14 has two slit wall faces 21, 22 which close, preferably tightly, the slit arrangement 14 in an unloaded initial state of the valve body 11.

The slit wall faces 21, 22 of the slit arrangement 14 are provided with a separating agent 23. The slit wall faces 21, 22 may be provided, in particular coated, with the separating agent 23 only partially or completely, i.e. across the entire surface.

Preferably, the separating agent 23 is present only on the slit wall faces 21, 22 of the slit arrangement 14. In other words, especially the head region 12 remaining and the valve casing 15 are preferably separating agent-free, i.e. free of the separating agent.

The separating agent 23 can advantageously prevent radiation-induced crosslinking of the elastically deformable material of the valve body 11, for example during gamma sterilization, beta sterilization, X-ray sterilization or electron beam sterilization (E-beam sterilization) of the connection device 1. This can significantly reduce or even completely avoid the risk of closing up of the slit arrangement 14.

The separating agent 23 comprises a solid, preferably biocompatible solid, or consists of a solid, preferably biocompatible solid. The solid is preferably a salt, particularly preferably sodium chloride.

Alternatively or in combination, the solid may preferably be a saccharide, particularly preferably glucose.

Alternatively or in combination, the solid may be a vitamin, particularly preferably vitamin C.

Aforementioned substances have been found to be particularly suitable with respect to avoiding closing up or healing of the slit arrangement 14 during radiation sterilization, such as in particular gamma radiation sterilization. In particular, a further advantage of these substances is that they do not affect any transparency of the valve body 11. In addition, the aforementioned substances can advantageously contribute to an increase in the ageing resistance of the valve body 11.

Once a tip of a component K to be connected to the connection nozzle 3 is brought from the outside to the connection nozzle 3, the tip comes into planar contact with the outer surface of the head region 12 and pushes the head region 12 into the interior of the cap 8. In this case, the slit arrangement 14 widens, and elastically deformed sections of the head region 12 are placed against the outside of the tip of the component K on further penetration of said tip. When the tip is disconnected and consequently removed outwardly, the head region 12 returns to the initial position according to FIG. 7.

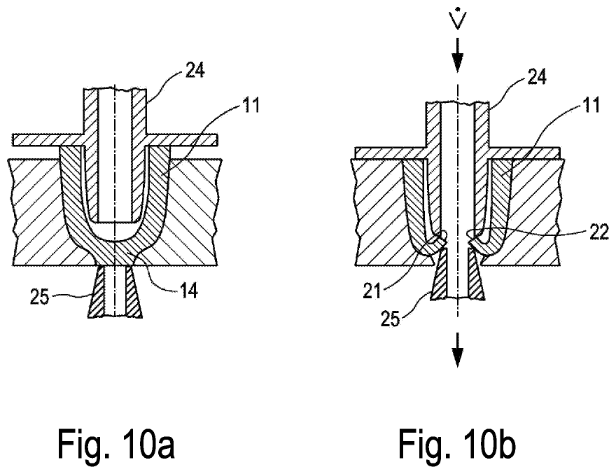

FIGS. 10a and b show schematically one embodiment of a method according to the invention.

The method first comprises coating the slit wall faces 21, 22 of the slit arrangement 14 with a pulverulent solid as separating agent. To this end, a valve body 11 is placed in a receptacle or directly on a nozzle 24. At the same time, the nozzle 24 can be brought closer using a hold-down device and/or a compression overlay until the valve body 11 is compressed. Furthermore, the valve body 11, as shown in FIGS. 10a and b, can be pressed against a Luer-like or cylindrical component 25, bringing about an at least partial opening of the valve body 11. In this case, the component 25, as shown in FIG. 10b, can enter somewhat.

The pulverulent solid is preferably applied by means of an air stream which is conducted through the partially or fully open valve body 11 in such a way that the air stream floods the slit wall faces 21, 22 of the slit arrangement 14 with the pulverulent solid. The pulverulent solid is transported by the air stream in the direction of the arrow (see FIG. 10b).

The air pressure for transporting the pulverulent solid to the slit wall faces 21, 22 of the slit arrangement 14 may be selected from 0.1 bar to 7 bar, preferably 0.5 bar to 3 bar. Furthermore, a volume flow time of 1 ms to 5 s, in particular 50 ms to 1 s, may be selected.

Preferably, the valve body 11 is compressed and thus opened in such a way that the inner lumen of the valve body 11 is not powder-coated.

1. A medical device comprising an elastically deformable functional part having an openable slit arrangement that widens or opens upon an elastic deformation of the elastically deformable functional part and recloses upon cessation of the elastic deformation, the slit arrangement comprising slit wall faces that are at least partially coated with a separating agent comprising or consisting of a solid.

2. The medical device according to claim 1, wherein the elastically deformable functional part is at least sectionally transparent.

3. The medical device according to claim 1, wherein the elastically deformable functional part is made of at least one of a silicone elastomer and a thermoplastic elastomer.

4. The medical device according to claim 1, wherein only the slit wall faces of the slit arrangement are at least partially coated with the separating agent.

5. The medical device according to claim 1, wherein the solid comprises a powder.

6. The medical device according to claim 1, wherein the solid has a particle size of <400 μm.

7. The medical device according to claim 1, wherein the solid is a salt.

8. The medical device according to claim 1, wherein the solid is a saccharide.

9. The medical device according to claim 1, wherein the solid is a vitamin.

10. The medical device according to claim 1, wherein the slit arrangement comprises a longitudinal slit arrangement.

11. The medical device according to claim 1, wherein the elastically deformable functional part comprises a valve body.

12. The medical device according to claim 1, wherein:
the elastically deformable functional part has a head region, and
the slit arrangement is formed along a transverse extent of the head region and extends centrally along a central longitudinal axis through the head region.

13. The medical device according to claim 1, wherein the medical device is configured for infusion therapy.

14. An elastically deformable functional part for a medical device, the elastically deformable functional part having an openable slit arrangement that widens or opens upon an elastic deformation of the elastically deformable functional part and recloses upon cessation of the elastic deformation, the slit arrangement comprising slit wall faces that are at least partially coated with a separating agent comprising or consisting of a solid.

15. A method for establishing sterilization resistance of the elastically deformable functional part according to claim 14, the method comprising the steps of:
a) coating or loading the slit wall faces of the slit arrangement with the separating agent; and
b) sterilizing the elastically deformable functional part.

16. The medical device according to claim 7, wherein the salt is an alkali metal salt.

17. The medical device according to claim 16, wherein the alkali metal salt is sodium chloride.

18. The medical device according to claim 8, wherein the saccharide is a monosaccharide.

19. The medical device according to claim 18, wherein the monosaccharide is glucose.

20. The medical device according to claim 9, wherein the vitamin is L-(+)-ascorbic acid.
